(19) United States
(12) Patent Application Publication
(10) Pub. No.: US 2025/0244292 A1
(43) Pub. Date: Jul. 31, 2025

(54) A MEASURING APPARATUS FOR INLINE MEASURING THE HOMOGENIZING DEGREE OF A FLUID PRODUCT IN A DISPERSING UNIT

(71) Applicant: GEA MECHANICAL EQUIPMENT ITALIA S.P.A., PARMA (IT)

(72) Inventors: Leonardo MAGGI, PARMA (IT); Annachiara TONELLI, COLLECCHIO (PARMA) (IT); Massimiliano BENASSI, FORNOVO di TARO (PARMA) (IT); AGNIESZKA BICZ, WROCLAW (PL); WIESLAW BICZ, WROCLAW (PL)

(21) Appl. No.: 18/694,788

(22) PCT Filed: Mar. 24, 2023
(86) PCT No.: PCT/IB2023/052951

§ 371 Date: Aug. 29, 2024

(30) Foreign Application Priority Data
Feb. 23, 2023 (IT) 102023000003195

(51) Int. Cl. G01N 29/024; G01N 29/22; G01N 33/02; G01N 33/15

(52) U.S. Cl. CPC G01N 29/024; G01N 29/222; G01N 29/228; G01N 33/02; G01N 33/15; G01N 2291/022

(57) ABSTRACT

A measuring apparatus for inline measuring the homogenizing degree of a fluid product in a dispersing unit, the measuring apparatus comprising: a tubular device arrangeable at an output of the dispersing unit, the tubular device having an internal cavity for the passage of the fluid product received from the dispersing unit; a plurality of ultrasound sensors mounted on the tubular device, wherein the internal cavity has a variable diameter across the length of the tubular device, the internal cavity having at least one first tract with a tapering development from an inlet of the tubular device towards an outlet of the tubular device.

### TECHNICAL FIELD

The present invention relates to a measuring apparatus for inline measuring the homogenizing degree of a fluid product in a dispersing unit. The invention proposed here is used in the food industry, in particular in the dairy sector, or in the chemical, pharmaceutical or cosmetic industry. The invention can also be used in manufacturing areas where homogenization is a step of the production process.

Consider, for example, the production of carbon-based nanostructured materials, such as graphene and carbon nanotubes or cellular breakdown of yeasts, algae, or microorganisms for the production of intracellular material.

### BACKGROUND ART

Even though in varying, currently known embodiments, a homogenizing apparatus comprises a high-pressure pump and a homogenizing valve which acts on the fluid products in order to:
crush the particles of the fluid to make their dimensions uniform, reducing the average size and the variance of the distribution in order to stabilize the product and to increase its shelf-life in the case of emulsions;
break the cell membranes in order to facilitate the extraction of the active ingredients in the case of pharmaceutical applications;
modify the structure of the particles in the case of chemical applications and cellulose or unicellular organisms.

For most products, the main parameters defining the level of homogenization are the average particle size and standard deviation. In the known solutions, the pressure of the homogenizing apparatus is adjusted as described here below.

First, the optimum average particle size and standard deviation for a given fluid product are experimentally determined. Then, it is applied a pressure value necessary to obtain particles showing the calculated parameters. Maintaining the pressure is achieved by one of the following methods:
periodically adjusting the pressure value by manual interventions;
using a feedback system which receives as input the pressure level detected in the fluid product and forces the output to follow a set point.

Both methods act directly on the homogenizing pressure.

Nevertheless, the homogenizing pressure cannot be considered a reliable real-time indicator of the actual homogenizing level. In fact, the pressure and the fluid product being equal, different homogenizing levels can be obtained, for example due to modifications occurring in the new product fed to the apparatus and/or due to wear of the components.

The known methods cannot track differences in the fluid product fed to the homogenizing apparatus.

In addition, the known methods do not consider changes caused in the fluid product by non-ideal components.

These situations are usually dealt with by designing a homogenizing apparatus able to assure a homogenizing level above a pre-established threshold, which corresponds to the most adverse conditions.

Having an oversized homogenizing apparatus raises risks of mechanical stress for some components, which may decrease their lifetime.

In addition, applying pressure values which are higher than requested is energy consuming.

Finally, applying pressure values which are higher than requested does not assure to reach the desired homogenizing level, as it causes an increase in temperature that may damage the fluid product. Part of the product could even be discarded due to thermal damage.

Document EP 1121973 A1 discloses a system for the supervision of a dispersion produced in a dispersing unit, in particular the homogenizing degree thereof, wherein it is proposed to couple at least one measurement sensor to the dispersing unit to achieve an in-line measurement of the homogenizing degree for the dispersion.

The sensor used in this document may be an ultrasonic sensor immersed in the fluid product or in contact with it. Thus, the sensor easily gets dirty and unreliable. In addition, it is more prone to be damaged by high temperature of the fluid product.

### DISCLOSURE OF THE INVENTION

In this context, the object of the present invention is to propose a measuring apparatus for inline measuring the homogenizing degree of a fluid product in a dispersing unit, which overcomes the problems of the prior art cited above.

In particular, the object of the present invention is to propose a measuring apparatus for inline measuring the homogenizing degree of a fluid product in a dispersing unit, which achieves a higher measurement reliability than the known solutions.

A further object of the present invention is to provide a measuring apparatus for inline measuring the homogenizing degree of a fluid product in a dispersing unit, which does not affect the fluid product.

Another object of the present invention is to propose a measuring apparatus for inline measuring the homogenizing degree of a fluid product in a dispersing unit, which is robust, less likely to wear over time and easy to clean.

Another object of the present invention is to propose a measuring apparatus for inline measuring the homogenizing degree of a fluid product in a dispersing unit, in which precautionary maintenance operations can be scheduled in a more efficient way.

The stated technical task and specified aims are substantially achieved by a measuring apparatus for inline measuring the homogenizing degree of a fluid product in a dispersing unit, the measuring apparatus comprising:
a tubular device arrangeable at an output of the dispersing unit, said tubular device having an internal cavity for the passage of the fluid product received from the dispersing unit;
a plurality of ultrasound sensors mounted on the tubular device, characterized in that the internal cavity has a variable diameter across the length of the tubular device, the internal cavity having at least one first tract with a tapering development from an inlet of the tubular device towards an outlet of the tubular device.

Preferably, the ultrasound sensors are distributed along the length of the tubular device.

According to one aspect of the invention, the first tract of the internal cavity is formed by a plurality of substantially cylindrical portions which are joined by frusto-conical portions, the cylindrical portions having corresponding diameters that decrease from the inlet towards the outlet of the tubular device.

Preferably, the internal cavity has a second tract originating from the first tract and ending at the outlet of the tubular device. In particular, the second tract has a tapering development from the outlet of the tubular device towards the first tract.

In particular, the second tract of the internal cavity consists of a frusto-conical portion connecting the first tract to the outlet of the tubular device.

According to one aspect of the invention, the tubular device comprises a first tube and a second tube. The first tube is coaxial with and internally arranged inside the second tube. The internal cavity is delimited by an internal surface of the first tube.

Preferably, the internal surface of the first tube partially has a tapering development from the inlet towards the outlet of the tubular device so as to delimit the corresponding tapering development of the first tract of the internal cavity.

According to one aspect of the invention, the ultrasound sensors are arranged between the first tube and the second tube.

Preferably, the first tube has a plurality of housings for receiving the ultrasound sensors. The housings are obtained as inward recesses of an external surface of the first tube.

In one embodiment of the invention, the ultrasound sensors are glued to the first tube.

In another embodiment of the invention, the ultrasound sensors are mounted externally to the second tube.

For example, the ultrasound sensors are screwed to the second tube.

Preferably, the first tube is made of a plastic material and the second tube is made of a metal material.

More preferably, the first tube is made of PEEK or PTFE.

The second tube is made of stainless steel.

According to an aspect of the invention, the tubular device comprises a heat exchanger for cooling down the ultrasonic sensors.

Preferably, the heat exchanger is a conduit for a cooling fluid. The conduit passes through the tubular device for its whole length and is parallel to a longitudinal axis of the tubular device.

### BRIEF DESCRIPTION OF DRAWINGS

Further characteristics and advantages of the present invention will more fully emerge from the non-limiting description of a preferred but not exclusive embodiment of a measuring apparatus for inline measuring the homogenizing degree of a fluid product in a dispersing unit, as illustrated in the accompanying drawings in which:

FIGS. 1 and 2 illustrate two different embodiments of a measuring apparatus for inline measuring the homogenizing degree of a fluid product, according to the present invention;

FIG. 3 shows the waveforms of measured signals received by four sensors in an experimental test carried out for choosing the best material for the first tube of the measuring apparatus;

FIG. 4 illustrates a block diagram of the measuring apparatus of FIGS. 1-2 at an output of a dispersing unit.

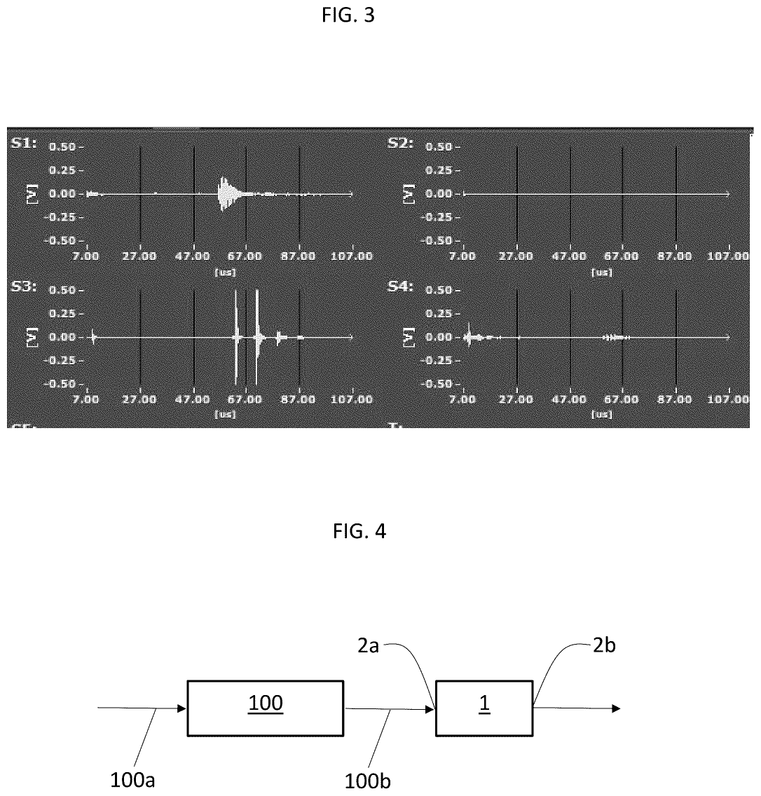

### DETAILED DESCRIPTION OF PREFERRED EMBODIMENTS OF THE INVENTION

With reference to the figures, the number 100 denotes a dispersion unit, The dispersion unit 100 has an input 100a and an output 101b for a fluid product.

For example, the dispersion unit 100 is a high-pressure homogenizer comprising a volumetric piston pump and a homogenizing valve arranged downstream of the volumetric piston pump.

In particular, the high-pressure homogenizer 100 operates with pressure up to 4000 bar.

Number 1 denotes a measuring apparatus for inline measuring the homogenizing degree of a fluid product in the dispersion unit 100.

The measuring apparatus 1 comprises a tubular device 2 arranged at the output 100b of the dispersion unit 100.

The tubular device 2 has an inlet 2a receiving the homogenized fluid product from the dispersion unit 100, and an outlet 2b.

The tubular device 2 has an internal cavity 3 for the passage of the fluid product received from the dispersion unit 100.

The internal cavity 3 extends from the inlet 2a to the outlet 2b of the tubular device 2.

The diameter of the internal cavity 3 at the inlet 2a and at the outlet 2b of the tubular device 2 is substantially the same.

Originally, the internal cavity 3 has a variable diameter across the length of the tubular device 2.

In particular, the internal cavity 3 has at least one first tract 31 with a tapering development from the inlet 2a towards the outlet 2b of the tubular device 2.

In other words, the first tract 31 has a convergent shape from the inlet 2a towards the outlet 2b of the tubular device 2.

Preferably, the first tract 31 of the internal cavity 3 is formed by a plurality of substantially cylindrical portions 310 which are joined by frusto-conical portions 311.

The cylindrical portions 310 have corresponding diameters that decrease from the inlet 2a towards the outlet 2b of the tubular device 2.

In the illustrated embodiment, the first tract 31 comprises five cylindrical portions 310 which are joined by four frusto-conical portions 311.

The internal cavity 3 preferably has a second tract 32 originating from the first tract 31 and ending at the outlet 2b of the tubular device 2.

The second tract 32 has a tapering development from the outlet 2b of the tubular device 2 towards the first tract 31.

In other words, the second tract 32 has a convergent shape from the outlet 2b of the tubular device 2 towards the first tract 31.

In particular, the second tract 32 consists of a frusto-conical portion 321 connecting the first tract 31 to the outlet 2b of the tubular device 2.

In particular, the last cylindrical portion 310 of the first tract 31 is connected to the frusto-conical portion 321 of the second tract 32.

The measuring apparatus 1 further comprises a plurality of ultrasound sensors 4 mounted on the tubular device 2 and configured to detect ultrasound waves.

According to the illustrated embodiment, the tubular device 2 comprises a first tube 5 and a second tube 6.

The first tube 5 is coaxial with and internally arranged inside the second tube 6.

The first tube 5 delimits the internal cavity 3. In particular, the internal cavity 3 is delimited by an internal surface 5a of the first tube 5.

In particular, the internal surface 5a of the first tube 5 partially has a tapering development from the inlet 2a towards the outlet 2b of the tubular device 2 so as to delimit the corresponding tapering development of the first tract 31 of the internal cavity 3.

According to one embodiment of the invention, the ultrasound sensors 4 are arranged between the first tube 5 and the second tube 6.

Preferably, the ultrasound sensors 4 are distributed along the length of the tubular device 2.

According to the embodiments illustrated herewith, the first tube 5 has a plurality of housings 7 for receiving the ultrasound sensors 4.

Preferably, the housings 7 are obtained as inward recesses of an external surface 5b of the first tube 5.

According to one embodiment of the invention, the ultrasound sensors 4 are glued to the first tube 5. In particular, they are placed inside the housings 7 and glued to the external surface 5b of the first tube 5.

According to another embodiment of the invention (not illustrated), the ultrasound sensors 4 are mounted externally to the second tube 6.

For example, the ultrasound sensors 4 are screwed externally to the second tube 6 so as to arrive close to the first tube 5.

Preferably, in this embodiment an ultrasound gel is deposited between the ultrasound sensors 4 and the first tube 5 for assuring the transmission of the signals.

Preferably, the measuring apparatus 1 has an electrical connector mounted on the second tube 6. The electrical connector serves for the connections of the ultrasound sensors 4.

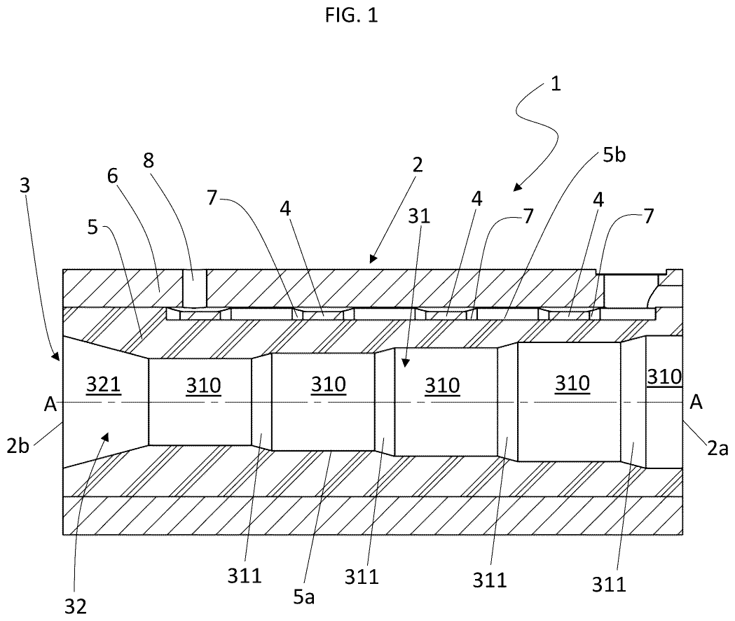

According to an embodiment of the invention, illustrated in FIG. 1, each cylindrical portion 310 is coupled to an ultrasound sensor 4. Said ultrasound sensor 4 is arranged inside a housing 7 obtained in a section of the first tube 5 surrounding the corresponding cylindrical portion 310.

In this embodiment, referred to as “Pulse Echo”, the ultrasound sensors 4 are configured to generate and detect ultrasound waves. In other words, the ultrasound sensors 4 act as emitters and receivers.

The waves generated by the ultrasound sensors 4 pass through the fluid product flowing in the internal cavity 3 and are reflected back by the second tube 6, which is made of metal.

Alternatively, reflectors may be mounted on a side opposite to the housings 7 with respect to the internal cavity 3 so as to increase the reflection of the ultrasound waves.

According to another embodiment of the invention, illustrated in FIG. 2, each cylindrical portion 310 is coupled to one of the ultrasound sensors 4 previously mentioned (which will be referred here as “receiver”), and to another ultrasound sensor 14 that is configured to generate ultrasound waves (and will be referred as “emitter”). The receivers 4 and the emitters 14 are respectively arranged in two housings 7 oppositely located with respect to the internal cavity 3. The two housings 7 are obtained in a section of the first tube 5 surrounding the corresponding cylindrical portion 310, but they are obtained on different sides with respect to the flow of the fluid product.

In this embodiment, referred to as “Through-Transmission”, the waves generated by the emitters 14 pass through the fluid product flowing in the internal cavity 3 and are detected by the receivers 4.

According to an aspect of the invention, the first tube 5 is made of a plastic material. Thus, ultrasound waves can pass through the first tube 5.

In a preferred embodiment, the first tube 5 is made of PEEK.

Alternatively, the first tube 5 is made of PTFE.

An experimental test has been carried out for choosing the best material for the first tube 5.

The test consisted in creating four different tubular sample portions, each formed by a different material for simulating the first tube 5.

Each sample portion has its own ultrasound sensor, which is a piezoelectric ceramic sensor with resonance frequency of 5 MHz. The ultrasound sensor is screwed to the corresponding sample portion.

These are the four tubular sample portions created for the test:
1^{st} sample portion: tube made of AISI 316L, with a thickness of 2 mm (from the internal cavity to the corresponding screwed sensor);
2^{nd} sample portion: tube made of PTFE, with a thickness of 10 mm (from the internal cavity to the corresponding screwed sensor);
3^{rd} sample portion: tube made of PEEK, with a thickness of 10 mm (from the internal cavity to the corresponding screwed sensor);
4^{th} sample portion: tube made of AISI 316L, with a thickness of 4 mm (from the internal cavity to the corresponding screwed sensor).

In practice, the sample portions are simplified versions of the tubular device 2 (with only the first tube 5), with a Pulse echo arrangement, where the ultrasound sensors act both as emitters and receivers.

The thicknesses of the first three samples are different. They have been chosen to obtain an adequate value of mechanical resistance to withstand an internal pressure of 40 bar inside the tube. The fluid inside the cavity is tap water at ambient temperature.

The 3^{rd} sample (PEEK) resulted the best choice since the signal after the first reflection is well distinguished over the noise.

PEEK is the best choice since it combines a high mechanical resistance with chemical inertia, suitability with food grade/pharmaceutical substances, resistance to high sterilization temperatures, transparency to ultrasonic waves.

In addition, the 4^{th} sample has been inserted to compare how the signal is transmitted in AISI 316L having a higher thickness (i.e., double) than the 1^{st} sample. In the 4^{th} sample the higher thickness of AISI 316L significantly attenuates the ultrasonic signal transmission, and the first reflection of the signal is affected by noise.

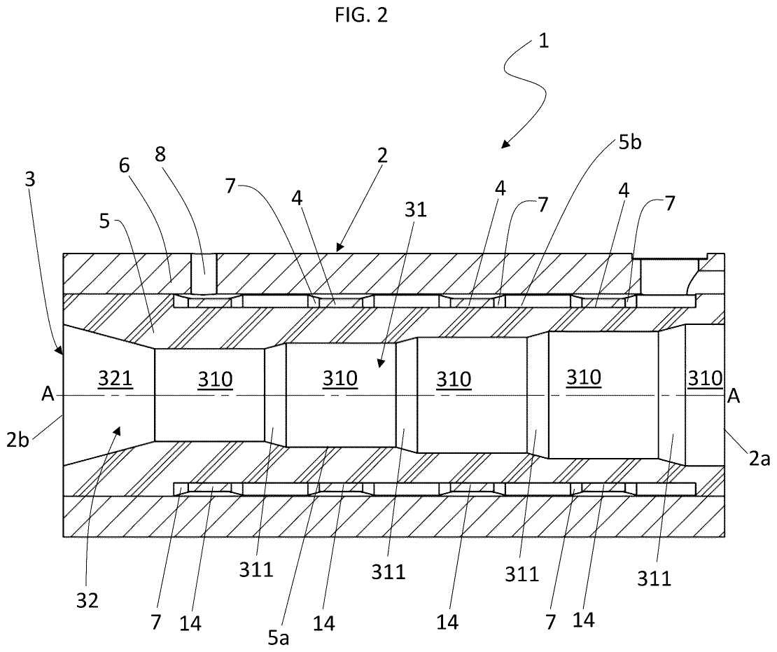

The measured signals received by the four sensors are reported in FIG. 3 for a quick comparison, where:
S1 refers to the 1^{st} sample portion;
S2 refers to the 2^{nd} sample portion;
S3 refers to the 3^{rd} sample portion;
S4 refers to the 4^{th} sample portion.

The second tube 6 is made of a metal material. For example, the second tube 6 is made of stainless steel. Thus, the ultrasound waves are reflected by the second tube 6 and maintained within the tubular device 2.

According to an aspect of the invention, the tubular device 2 comprises a heat exchanger 8 for cooling down the ultrasonic sensors 4.

Preferably, the heat exchanger 8 is a conduit for the passage of a cooling fluid.

Preferably, the conduit 8 passes through the tubular device 2 for its whole length and has a main tract that is parallel to a longitudinal axis A-A of the tubular device 2.

According to one embodiment of the invention, the conduit 8 has an inlet obtained in the second tube 6 in correspondence of the housing 7 which is located closer to the inlet 2a of the tubular device 2, and an outlet obtained in the second tube 6 in correspondence of the housing 7 which is located closer to the outlet 2b of the tubular device 2.

Alternatively, the inlet of the conduit 8 is closer to the outlet 2b of the tubular device 2, and the outlet of the conduit 8 is closer to the inlet 2a of the tubular device 2. In particular, the conduit 8 is fed with air having a temperature of 5°-40° C.

The air flowing within the conduit 8 serves for maintaining under control the temperature of the ultrasound sensors 4, independently from the temperature of the fluid product flowing in the internal cavity 3.

This is advantageous both during the normal working of the dispersing unit 100 and during sterilization cycles, where the tubular device 2 can reach high temperature (for example 130° C.) which may affect the reliability of the ultrasound sensors 4 or even damage them.

In addition, in the embodiment in which the ultrasound sensors 4 are glued to the first tube 5, a high temperature could unglue the sensors 4 from the first tube 5, and an air layer could originate between the sensors 4 and the first tube 5, thus interrupting signal transmissions.

The ultrasound sensors 4 are configured to receive ultrasound waves having a frequency comprised between 2 MHz and 20 MHz.

Preferably, the ultrasound sensors 4 are chosen to work at different frequencies so as to detect the ultrasound waves passing in fluid products with different acoustic impedances.

The signal detected by the ultrasound sensors 4 may be used to control the pressure of the fluid product in the dispersion unit 100.

The characteristics of the measuring apparatus for inline measuring the homogenizing degree of a fluid product in a dispersing unit, according to the present invention emerge clearly from the above description, as do the advantages.

Since the ultrasound waves are distributed along the whole length of the tubular device, detection is simultaneously carried out in multiple measurement points of the flow of fluid product.

Thus, this detection is more reliable than detecting in a single point or a single specimen of fluid product. Redundancy is also advantageous in case of damage of one or more sensors.

In addition, arranging the ultrasound sensors on the external surface of the first tube avoids a direct contact with the fluid product which flows in the internal cavity. This results in a beneficial hygienic design of the tubular device.

In addition, in the embodiment in which the ultrasound sensors are glued to the first tube, there is substantially no air in the housings for the ultrasound sensors. This assures a good quality of signals, since air would attenuate ultrasound waves.

Furthermore, in the embodiment in which the ultrasound sensors are glued to the first tube, the ultrasound sensors are substantially maintained in their positions during the assembling and disassembling operations of the tubular device. This is assured by their arrangement in the inward recesses of the external surface of the first tube. Indeed, this arrangement also protects the ultrasound sensors from damages.

In the embodiment in which the ultrasound sensors are glued to the first tube, the ultrasound sensors are not mechanically coupled to the second tube, which is made of stainless steel. Thus, there is no risk of accumulation of particles (i.e., dust) between the second tube and the ultrasound sensors. In addition, the second tube is almost smooth, with only one electrical connector. The external surface of the second tube is thus easy to clean.

1. A measuring apparatus for inline measuring the homogenizing degree of a fluid product in a dispersing unit, said measuring apparatus comprising:
a tubular device arrangeable at an output of the dispersing unit, said tubular device having an internal cavity for the passage of the fluid product received from the dispersing unit;
a plurality of ultrasound sensors mounted on the tubular device, wherein said internal cavity has a variable diameter across the length of the tubular device, the internal cavity having at least one first tract with a tapering development from an inlet of the tubular device towards an outlet of the tubular device.

2. The measuring apparatus according to claim 1, wherein the first tract of the internal cavity is formed by a plurality of substantially cylindrical portions which are joined by frusto-conical portions, said cylindrical portions having corresponding diameters that decrease from the inlet towards the outlet of the tubular device.

3. The measuring apparatus according to claim 2, wherein the internal cavity has a second tract originating from the first tract and ending at the outlet of the tubular device, said second tract having a tapering development from the outlet of the tubular device towards said first tract.

4. The measuring apparatus according to claim 3, wherein the second tract of the internal cavity consists of a frusto-conical portion connecting the first tract to the outlet of the tubular device.

5. The measuring apparatus according to claim 1, wherein the tubular device comprises a first tube and a second tube, the first tube being coaxial with and internally arranged inside the second tube, said internal cavity being delimited by an internal surface of the first tube.

6. The measuring apparatus according to claim 5, wherein the internal surface of the first tube partially has a tapering development from the inlet towards the outlet of the tubular device so as to delimit the corresponding tapering development of the first tract of the internal cavity.

7. The measuring apparatus according to claim 5, wherein the ultrasound sensors are arranged between the first tube and the second tube.

8. The measuring apparatus according to claim 7, wherein the first tube has a plurality of housings for receiving said ultrasound sensors, said housings being obtained as inward recesses of an external surface of the first tube.

9. The measuring apparatus according to claim 7, wherein the ultrasound sensors are glued to the first tube.

10. The measuring apparatus according to claim 5, wherein the ultrasound sensors are mounted externally to the second tube.

11. The measuring apparatus according to claim 10, wherein the ultrasound sensors are screwed to the second tube.

12. The measuring apparatus according to claim 5, wherein the first tube is made of a plastic material and the second tube is made of a metal material.

13. The measuring apparatus according to claim 12, wherein the first tube is made of PEEK or PTFE.

14. The measuring apparatus according to claim 12, wherein the second tube is made of stainless steel.

15. The measuring apparatus according to claim 1, wherein the tubular device comprises a heat exchanger for cooling down the ultrasonic sensors.

16. The measuring apparatus of claim 15, wherein the heat exchanger is a conduit for a cooling fluid, said conduit passing through the tubular device for its whole length and being parallel to a longitudinal axis of the tubular device.

17. The measuring apparatus according to claim 1, wherein the ultrasound sensors are distributed along the length of the tubular device.
